(19) United States
(12) Patent Application Publication
(10) Pub. No.: US 2025/0359936 A1
(43) Pub. Date: Nov. 27, 2025

(54) AUTOMATED SEGMENTATION FOR ACL REVISION OPERATIVE PLANNING

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Alyssa ANDRADE, Dedham, MA (US); Nathan Anil NETRAVALI, Littleton, MA (US); Michel Gonçalves ALMEIDA ANTUNES, Coimbra (PT); Inês Dinis FÉLIX, Lisbon (PT)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 18/873,467

(22) PCT Filed: Aug. 2, 2023
(86) PCT No.: PCT/US2023/029281

§ 371 Date: Dec. 10, 2024

Related U.S. Application Data
(60) Provisional application No. 63/401,837, filed on Aug. 29, 2022.

(51) Int. Cl. A61B 34/10; A61B 6/50; G06T 7/00; G06T 7/11; G06T 7/136; G06T 11/00; G06T 17/00

(52) U.S. Cl. CPC A61B 34/10; G06T 7/0012; G06T 7/11; G06T 7/136; G06T 11/00; G06T 17/00; A61B 6/505; A61B 2034/105; G06T 2207/10081; G06T 2207/10088; G06T 2207/30008; G06T 2210/41

(57) ABSTRACT

Disclosed are systems and methods for a computerized framework that provides novel mechanisms for the automatic identification of existing tunnels and hardware, which can be used for compiling of a preoperative and/or intraoperative plan for an anterior cruciate ligament (ACL) revision procedure. The operative plan, among other benefits, automatically avails surgeons with capabilities to locate the tunnels physically, and guides them in their revision ACL reconstruction procedure. According to some embodiments, the disclosed framework can generate synthetic ACL reconstruction CT images from CT images of patients without previous primary ACL reconstruction. The framework can generate realistic ACL reconstruction CTs, which can be used as input for training machine learning or deep learning models. Moreover, this can improve the accuracy, robustness and generalization capacity (e.g., identification of tunnels and hardware in MRIs and CTs) of the machine learning and deep learning based models for ACL tunnel segmentation.

### CROSS-REFERENCE TO RELATED APPLICATIONS

This application claims the benefit of U.S. Prov. App. 63/401,837 filed Aug. 29, 2022 and titled “Automated Segmentation for ACL Revision Operative Planning.”

### TECHNICAL FIELD

The present disclosure generally relates to preoperative and intraoperative surgical analysis and processing, and more particularly, to computerized methodologies for automatically generating and providing Anterior Cruciate Ligament (ACL) information for an ACL revision procedure.

### BACKGROUND

The ACL is one of the four main ligaments in the knee that connects the femur to the tibia. ACL reconstruction is one of the most common surgeries in orthopedics with around 100,000 ACL reconstructions per year in the United States. However, the failure rate of these procedures varies from 10 to 25 percent, and many patients must undergo a revision ACL reconstruction. Revision ACL is a procedure to reconstruct the ACL after primary ACL reconstruction has failed.

### SUMMARY

ACL revision typically involves operative planning in order to account for existing tunnels and hardware that were already in the joint from the primary (and failed) ACL reconstruction. Revision ACL reconstruction is more difficult than primary ACL reconstruction due to the existence of these remnants from the initial procedure. Thus, ACL revision procedures require planning and execution to locate such tunnels and hardware, and determine if (and where) there are any abnormal tunnels (e.g., abnormal tunnel widening, and the like, for example).

Thus, there are many challenges associated with revision ACL procedures because the surgeon needs to account for the preceding ACL procedure. Surgeons typically order computed tomography (CT) scans that allow them to visually locate the tunnels and hardware relative to the anatomy. However, it can be challenging to determine positioning for the new tunnels while accounting for the existing tunnels and hardware. If the new tunnels are placed incorrectly, there can be issues with improper graft fixation and tensioning. If the previous tunnel placement was grossly malpositioned, it can be avoided during the new tunnel placement. Partially overlapping tunnels can require a two stage procedure in which bone is grafted first to fill any bony voids and then a second procedure is performed to perform the reconstruction.

Moreover, hardware removal is another challenge in revision ACL procedures. There are two types of hardware typically used, metal and bioabsorbable. Metal hardware that conflicts with new tunnel position must be removed. Bioabsorbable screws can remain in place and can be drilled through if needed.

Overall, it is important for surgeons to understand the locations of the tunnels and hardware from the primary reconstruction for operative planning to determine the proper locations of new tunnels and hardware.

Conventional methods typically involve surgeons analyzing the patient's condition before surgery using magnetic resonance imaging (MRI) and CT images. However, there are shortcomings to such approaches that are tied to inaccurate readings and/or inaccurate imagery of existing bone and ligament conditions.

The disclosed systems and methods, therefore, provide a novel framework that provides a machine learning (ML) algorithm that enables a novel technical solution for the automatic identification of existing tunnels and hardware, which can be used/leveraged when preparing a preoperative and/or intraoperative plan for an ACL revision procedure. The preoperative and intraoperative plan(s), among other benefits, avails surgeons with capabilities to locate the tunnels physically, and guides them in their revision ACL reconstruction procedure.

According to some embodiments, the disclosed framework can navigate situations that involve or are resultant from improper initial ACL procedures (e.g., the tunnels were incorrectly located and/or placed or include abnormal tunnel-widening, for example, which increases the chances of an unsuccessful ACL revision and complete failure of the procedure). As such, according to some embodiments of the instant disclosure, the disclosed framework can deploy the ML algorithm where it is configured to generate synthetic ACL reconstruction CT images from CT images of patients without previous primary ACL reconstruction. As discussed in more detail below, this enables the generation of realistic looking ACL reconstruction CTs, which can be used as input for training machine learning or deep learning models. Moreover, this can improve the accuracy, robustness and generalization capacity (e.g., identification of tunnels and hardware in MRIs and CTs) of the machine learning and deep learning based models for ACL tunnel segmentation.

The disclosed systems and methods provide a computerized framework that addresses current shortcomings in the existing technologies, inter alia, by providing novel mechanisms for automatically generating and providing ACL information for an ACL revision procedure.

In accordance with one or more embodiments, the present disclosure provides a non-transitory computer-readable storage medium for carrying out the above mentioned technical steps. The non-transitory computer-readable storage medium has tangibly stored thereon, or tangibly encoded thereon, computer readable instructions that when executed by a device, cause at least one processor to perform a method for providing novel mechanisms for automatically generating and providing ACL information for an ACL revision procedure.

In accordance with one or more embodiments, a system is provided that comprises one or more computing devices and/or apparatus configured to provide functionality in accordance with such embodiments. In accordance with one or more embodiments, functionality is embodied in steps of a method performed by at least one computing device and/or apparatus. In accordance with one or more embodiments, program code (or program logic) executed by a processor(s) of a computing device to implement functionality in accordance with one or more such embodiments is embodied in, by and/or on a non-transitory computer-readable medium.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary data flow according to some embodiments of the present disclosure;

FIG. 4 depicts a non-limiting example embodiment of the disclosed technology according to some embodiments of the present disclosure;

FIG. 5 depicts a non-limiting example embodiment of the disclosed technology according to some embodiments of the present disclosure;

FIG. 6 depicts a non-limiting example embodiment of the disclosed technology according to some embodiments of the present disclosure;

FIG. 7 illustrates an exemplary data flow according to some embodiments of the present disclosure;

FIG. 8 depicts a non-limiting example embodiment of the disclosed technology according to some embodiments of the present disclosure;

FIG. 9 depicts a non-limiting example embodiment of the disclosed technology according to some embodiments of the present disclosure; and

FIG. 10 is a block diagram illustrating a computing device showing an example of a device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Unless limited otherwise, the terms “connected,” “coupled,” and “mounted,” and variations thereof herein are used broadly and encompass direct and indirect connections, couplings, and mountings. In addition, the terms “connected” and “coupled”” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present disclosure, and are not intended to limit the structure of the exemplary embodiments of the present disclosure to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

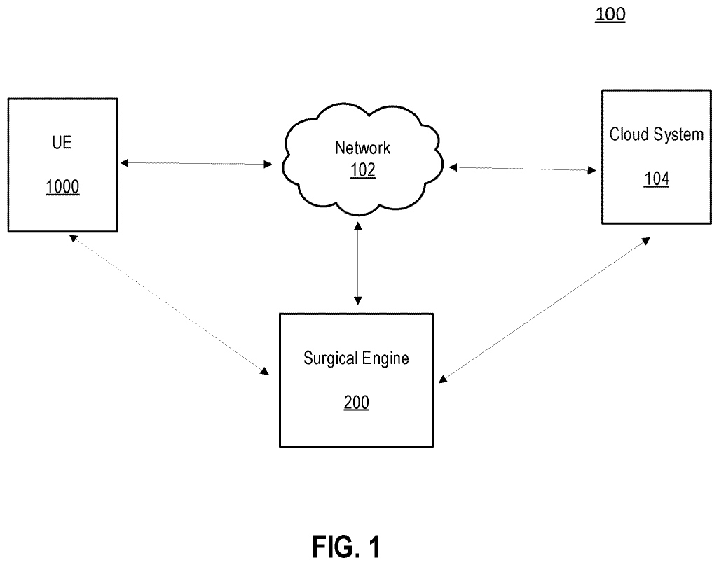

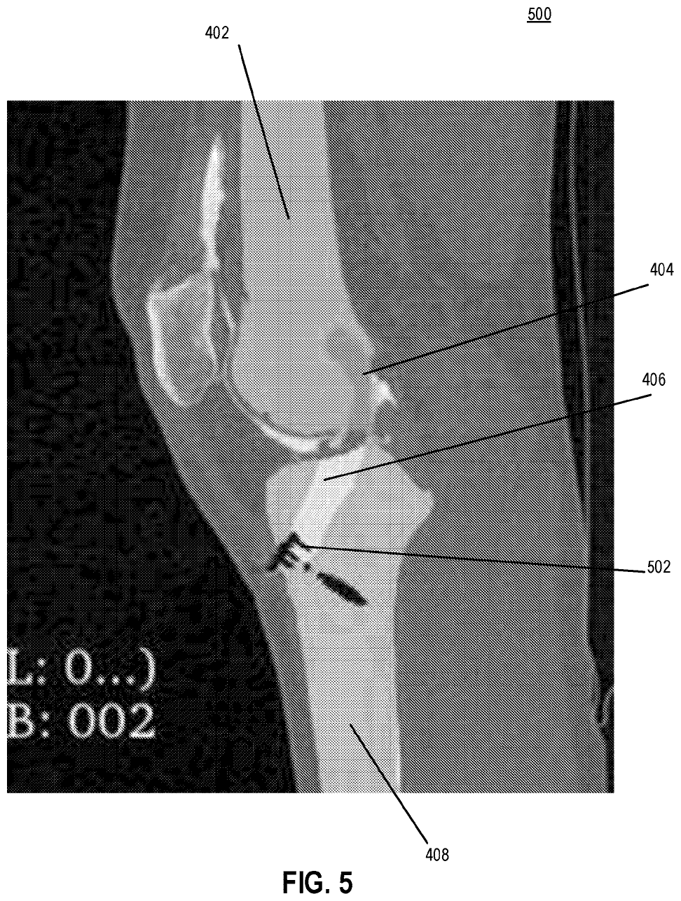

With reference to FIG. 1, system (or framework) 100 is depicted which includes UE 1000 (e.g., a client device), network 102, cloud system 104 and surgical engine 200. UE 1000 can be any type of device, such as, but not limited to, a mobile phone, tablet, laptop, personal computer, sensor, Internet of Things (IoT) device, autonomous machine, and any other device equipped with a cellular or wireless or wired transceiver. In some embodiments, as discussed above, UE 1000 can also be a medical device, or another device that is communicatively coupled to a medical device that enables reception of readings from sensors of the medical device. For example, in some embodiments, UE 1000 can be a user's smartphone (or office/hospital equipment, for example) that is connected via WiFi, Bluetooth Low Energy (BLE) or NFC, for example, to a peripheral neuromodulation device. Thus, in some embodiments, UE 1000 can be configured to receive data from sensors associated with a medical device, as discussed in more detail below. Further discussion of UE 1000 is provided below at least in reference to FIG. 5.

Network 102 can be any type of network, such as, but not limited to, a wireless network, cellular network, the Internet, and the like (as discussed above). As discussed herein, network 102 can facilitate connectivity of the components of system 100, as illustrated in FIG. 1.

Cloud system 104 can be any type of cloud operating platform and/or network based system upon which applications, operations, and/or other forms of network resources can be located. For example, system 104 can correspond to a service provider, network provider and/or medical provider from where services and/or applications can be accessed, sourced or executed from. In some embodiments, cloud system 104 can include a server(s) and/or a database of information which is accessible over network 102. In some embodiments, a database (not shown) of system 104 can store a dataset of data and metadata associated with local and/or network information related to a user(s) of UE 1000, patients and the UE 1000, and the services and applications provided by cloud system 104 and/or surgical engine 200.

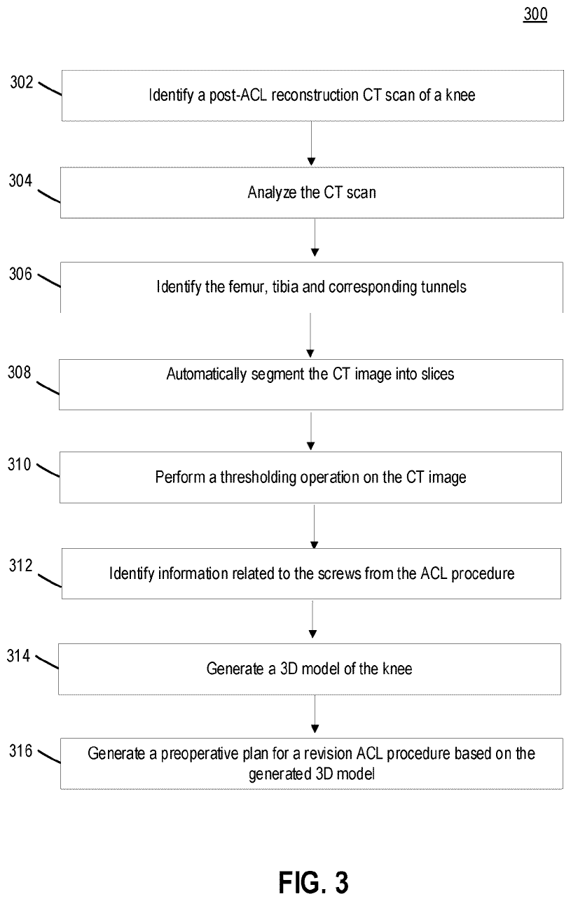

Surgical engine 200, as discussed below in more detail, includes components for automatically generating and providing Anterior Cruciate Ligament (ACL) information. Embodiments of how engine 200 operates and functions, and the capabilities it includes and executes, among other functions, are discussed in more detail below in relation to FIGS. 3-7.

According to some embodiments, surgical engine 200 can be a special purpose machine or processor and could be hosted by a device on network 102, within cloud system 104 and/or on UE 1000. In some embodiments, engine 200 can be hosted by a peripheral device connected to UE 1000 (e.g., a medical device, as discussed above).

According to some embodiments, surgical engine 200 can function as an application provided by cloud system 104. In some embodiments, engine 200 can function as an application installed on UE 1000. In some embodiments, such application can be a web-based application accessed by UE 1000 over network 102 from cloud system 104 (e.g., as indicated by the connection between network 102 and engine 200, and/or the dashed line between UE 1000 and engine 200 in FIG. 1). In some embodiments, engine 200 can be configured and/or installed as an augmenting script, program or application (e.g., a plug-in or extension) to another application or program provided by cloud system 104 and/or executing on UE 1000.

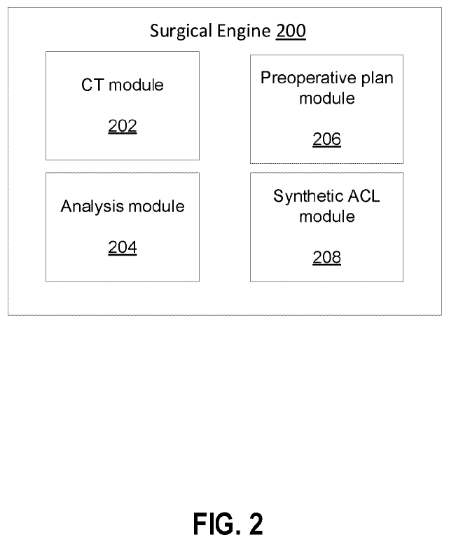

As illustrated in FIG. 2, according to some embodiments, surgical engine 200 includes CT module 202, analysis module 204, operative plan module 206 and synthetic ACL module 208. One of skill in the art would readily recognize and understand that module 206 includes references to, and applicability to preoperative, intraoperative and/or post-operative planning. Moreover, it should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning to FIG. 3, depicted is Process 300 which details non-limiting example embodiments of the disclosed framework's computerized operations for automatically generating and providing Anterior Cruciate Ligament (ACL) information. According to some embodiments, as discussed herein, the disclosed framework can automatically identify/segment screws and tunnels which will serve as an input to preoperative and/or intraoperative planning. According to some embodiments, the disclosed framework, which involves the execution of a machine learning (ML) algorithm, as discussed below, automatically segments the femur, tibia, femoral tunnel and tibial tunnel(s) when given a post-ACL reconstruction CT scan. As discussed below, the screw can be located by thresholding, via a predetermined range (e.g., 2000 Hounsfield Units (HU)-3000 HU), which can involve the identification of specific types of materials—for example, only metal and not any soft tissue or bone. The disclosed framework can then prepare, generate and output for display a three-dimensional (3D) model of the knee which can be used as the basis for preoperative and/or intraoperative planning for an ACL revision procedure.

According to some embodiments, Step 302 of Process 300 can be performed by CT module 202 of surgical engine 200; Steps 304-312 can be performed by analysis module 204; and Steps 314-316 can be performed by operative plan module 206.

Process 300 begins with Step 302 where engine 200 identifies a post-ACL reconstruction CT scan of a knee of a patient. According to some embodiments, Step 302 can involve the capture of the CT scan and storage in an associated database of engine 200. In some embodiments, the CT scan can be a digital image file.

It should be understood that while the discussion herein will focus on images captured via CT scanning, it should not be construed as limiting, as other known or to be known forms of digital image capturing techniques (e.g., magnetic resonance imaging (MRI), compressive sensing (CS) and the like) can be utilized without departing from the scope of the instant disclosure.

In Step 304, engine 200 analyzes the CT scan. In some embodiments, Step 304 can involve engine 200 executing any type of known or to be known computational analysis technique, algorithm, software or mechanism that can analyze the CT scan and segment the digital image file according to a set of criteria, such as, but not limited to, a medical imaging interaction toolkit-generate models (MITK-GEM) software, Efficient Residual Factorized ConvNet (ERFNet), UNet, ENet, computer vision, neural network, region-based or edge-based segmentation, and the like, or some combination thereof.

Thus, Step 304 can involve the analysis of the CT scan obtained in Step 302, which, in Step 306, results in the identification of the digital representations of the femur, tibia and corresponding tunnels (or tunnel) from the initial ACL reconstruction procedure.

In Step 308, based on the analysis from Step 304 and identification of the femur, tibia and tunnel(s) from Step 306, engine 200 can then segment the CT image into respective slices. The segmentation into slices involves the identification of the corresponding regions in each of those slices. Accordingly, the segmentation occurring in Step 308 can be based on the computational analysis techniques discussed above in relation to at least Step 304.

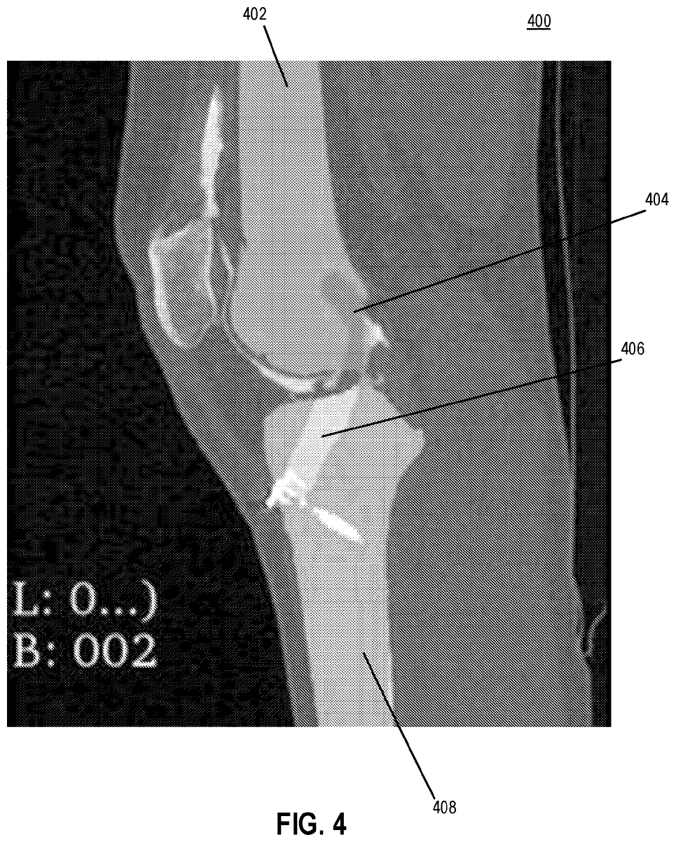

Turning to FIG. 4, CT image 400 is displayed, which depicts femur 402 and tibia 408. Image 400 further depicts tunnels 404 and 406. According to some embodiments, based on the segmentation from Steps 302-308, discussed above, engine 200 can identify femur 402, tunnel 404 that is the tunnel within femur 402, tibia 408 and tunnel 406 that is within tibia 408.

Turning back to FIG. 3, Process 300 continues from Step 308 to Step 310 where engine 200 performs a thresholding operation on the CT image. Thus, having identified the bones (e.g., femur and tibia) and the existing tunnels, as discussed supra, engine 200 can perform Step 310's thresholding operation, which results in Step 312 where information related to the screws from the initial ACL procedure is identified.

According to some embodiments, engine 200's execution of Steps 310-312 result in the determination of a location and/or quantity of screws (which were used in the initial ACL procedure). According to some embodiments, the identity of screws in the CT scan and their location therein can be a result of segmenting via a thresholding operation, which can be based on a predetermined range of HUs. In some embodiments, such thresholding can be performed in accordance with a bimodal histogram where segmentation (e.g., to identify a screw) can be based on a threshold range (e.g., a range of HU). In some embodiments, the range can be selected so as to enable identification of a particular material (e.g., only identify metal, and do not identify any soft tissue or bone). By way of a non-limiting example, a screw(s) within a CT scan can be located by performing a thresholding operation according to a range of 2000 HU-3000 HU.

According to some embodiments, the location and/or quantity of screws, as discussed above, can be determined by utilizing any of the machine learning and/or deep learning techniques discussed above at least in relation to Step 304.

Turning to FIG. 5, CT image 500 is depicted, which an example of a CT image that was subject to the thresholding of Steps 310-312. CT image 500 depicts the femur 402, tunnel 404, tibia 408, tunnel 406 and screws 502.

Turning back to FIG. 3, Process 300 proceeds from Step 312 to Step 314 where, having detected the femur, tibia and the tunnels (from, e.g., Step 308), and screws (from, e.g., Step 312), engine 200 then operates to generate a 3D model of the knee. According to some embodiments, 3D modelling of Step 314 can be performed using any type of known or to be known algorithm, technique or mechanism, such as, but not limited to, computer vision, neural network, artificial intelligence (AI), 3D ML algorithm, and the like. For example, the 3D model can be generated (or rendered) based on engine 200 executing a program such as, but not limited to, ACL PRIME or another form of MITK-GEM.

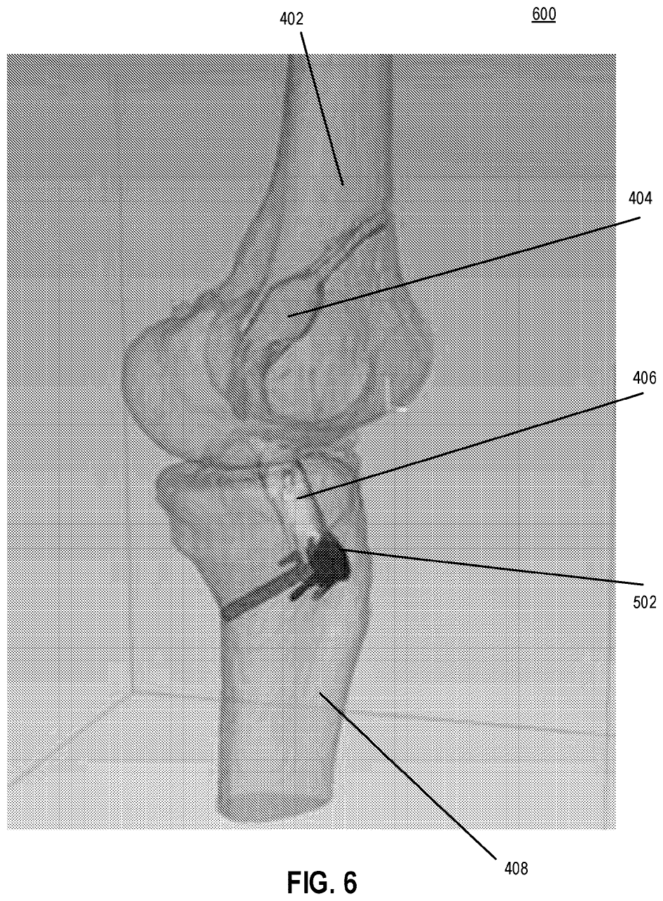

Turning to FIG. 6, 3D model 600 is depicted, which is an example of the generated 3D model from Step 314. 3D model 600 depicts 3D renderings of the femur 402, tunnel 404, tibia 408, tunnel 406 and screws 502.

Turning back to FIG. 3, Process 300 proceeds from Step 314 to Step 316 where engine 200 generates an operative (e.g., preoperative and/or intraoperative) plan for a revision ACL procedure based on the generated 3D model. The operative plan can be a data file, information file, encrypted or secure file or any other type of electronic document, item, file or object that stores information about the patient for a revision ACL procedure, which can include, but it not limited to, the 3D model, the segmentation from Step 308, thresholding from Step 312, the CT image, and the like, or some combination thereof.

As such, the disclosed methodology of Process 300 enables an accurate portrayal of bone positions, and where tunnel and screws from a previous ACL procedure are located so that an accurate operative plan can be compiled for an ACL revision procedure. In some embodiments, the operative plan can be compiled from the determined information from Process 300 automatically via any type of known or to be known computational analysis-based machine learning or artificial intelligence algorithm or software; and in some embodiments, a surgeon (or other type of medical professional) can leverage the determined information from Process 300 as part of a created operative plan. As such, the operative plan can be utilized during a preoperative stage of an ACL revision procedure and/or an intraoperative stage of an ACL revision procedure.

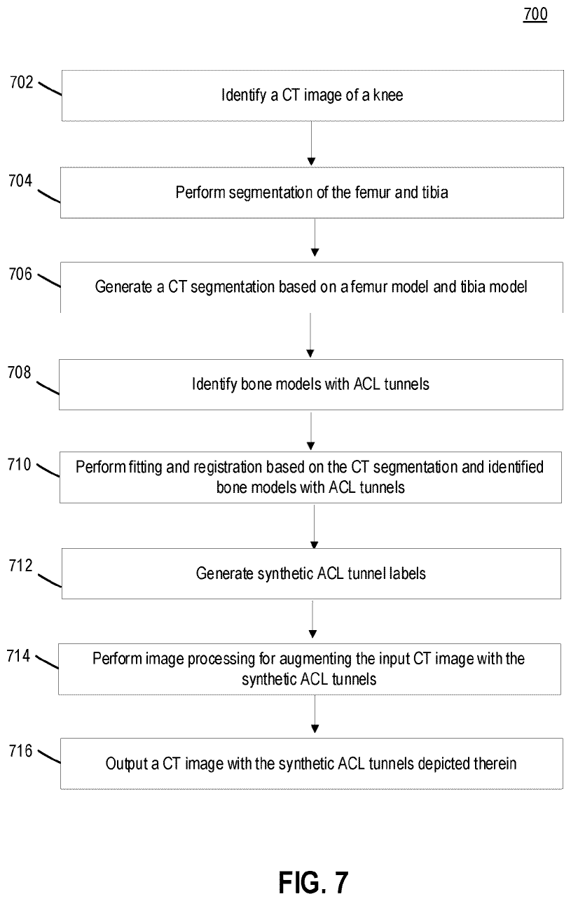

Turning to FIG. 7, Process 700 details non-limiting example embodiments of the disclosed framework's employment of an ML algorithm that is configured to generate synthetic ACL reconstruction CT images from CT images of patients without previous primary ACL reconstruction. As discussed herein, this enables the generation of realistic looking ACL reconstruction CTs, which in some embodiments, can be utilized for model training and/or operative (e.g., preoperative and/or intraoperative) planning, as discussed above in relation to at least FIG. 3.

According to some embodiments, Step 702 of Process 700 can be performed by CT module 202 of surgical engine 200; Steps 704-710 can be performed by analysis module 204; and Steps 712-716 can be performed by synthetic ACL module 208.

Process 700 begins with Step 702 engine 200 identifies a CT image of a knee. This can be performed in a similar manner as discussed above in relation to Step 302 of Process 300 of FIG. 3. In some embodiments, as mentioned above, the CT image identified in Step 702, however, captures a digital representation of a patient's knee that was not subject to primary ACL reconstruction (e.g., ACL tunnels from a previous or primary ACL procedure are not present).

It should be understood that while the discussion herein will focus on images captured via CT scanning, it should not be construed as limiting, as other known or to be known forms of digital image capturing techniques (e.g., MRI, CS and the like) can be utilized without departing from the scope of the instant disclosure.

In Step 704, engine 200 performs segmentation of the femur and tibia depicted within the CT image. In Step 706, engine 200 generates a CT segmentation based on the segmentation from Step 704. According to some embodiments, the computations from Steps 704-706 performed by engine 200 can be performed in a similar manner as discussed above in relation to Steps 304-308, where the slices of the femur and tibia from the CT image are identified.

In Step 708, engine 200 identifies bone models with ACL tunnels. According to some embodiments, the bone models can be Statistical Shape Models (SSMs). As understood by those of skill in the art, SSMs are geometric models that describe a collection of semantically similar objects that account for 3D objects and ranges of variations in shapes and sizes. For purposes of this disclosure, the SSMs provide a set of femur models, tibia models and ACL femur and tibia tunnel models, and/or a combination thereof. For example, the SSM provides a model of a knee that has had a primary ACL procedure.

Thus, in Step 708, engine 200 can search for (within an SSM database, or similar database that hosts SSM models) and identify a SSM that corresponds to the femur and tibia depicted in the CT image (from Step 702). For example, engine 200 can query a database using information from the CT segmentation (from Step 706) to identify a set of SSMs.

In Step 710, engine 200 performs fitting and registration based on the CT segmentation (from Step 706) and the identified SSMs (from Step 708). Accordingly, Step 710 can involve engine 200 aligning the SSM with the CT segmentation so that ACL tunnels from the SSM can be transferred in CT space to the CT segmentation (and subsequently to the CT image).

In some embodiments, the alignment of Step 710 can further or alternatively be based on anatomical reference frames (ARFs) of the knee, which can be derived from the CT image.

In Step 712, based on the fitting and registration (e.g., alignment) from Step 710, engine 200 can generate synthetic ACL tunnel labels. The labels can correspond to the SSM which can be used for synthetic generation of a CT image, as discussed below. In some embodiments, the labels can indicate, but are not limited to, a location, size, shape and/or quantity of the tunnels, and positions within specific bones (e.g., femur or tibia).

In some embodiments, it should be understood that while the discussion herein (e.g., for Step 712) can be utilized for synthetic tunnel labels, it should not be so limiting, as any other type of discernable information for a knee and/or ACL procedure can be determined, generated and labeled, such as, but not limited to, synthetic images with screws, the posterior cruciate ligament (PCL) tunnels, osteophytes, fractures, surgical hardware, and the like, or some combination thereof.

In Step 714, engine 200 performs image processing for augmenting the input CT image (from Step 702) within the synthetic ACL tunnels. According to some embodiments, the image processing can involve projecting the ACL tunnels (based on their labels) into the two-dimensional (2D) slices from the CT segmentation. The intersection of the CT slices and the 3D tunnel models can correspond to the tunnel segmentations. In some embodiments, the ACL tunnel regions can then filled with image patches that digitally represent the tunnels (and are configured to look like the tunnels). In some embodiments, the image patches can be computed by randomly selecting an image patch in the input CT not belonging to a bone region nor the background.

In some embodiments, instead of using image patches from the input CT for covering the tunnel region, either a SSM for image intensities or Generative Adversarial Networks (GANs) can be utilized.

And, in Step 714, engine 200 can output a CT image with the synthetic ACL tunnels depicted therein. In some embodiments, the output CT image in Step 714 can be a newly generated CT image, and in some embodiments, the output CT image can be an augmented version of the CT image from Step 702. In some embodiments, the output CT image (and the corresponding information determined, derived or otherwise identified from the preceding steps of Process 700) can be utilized for preoperative and/or intraoperative planning and/or training engine 200 to perform Process 300.

Accordingly, as discussed above, Process 700 can additionally be utilized to synthetically augment a CT image with synthetic screws, PCL tunnels, osteophytes, fractures, surgical hardware, and the like, or some combination thereof, in a similar manner using SSMs that have such corresponding information. One of skill in the art would readily understand that such additional and/or alternative synthetic augmentation would not depart from the scope of the instant disclosure.

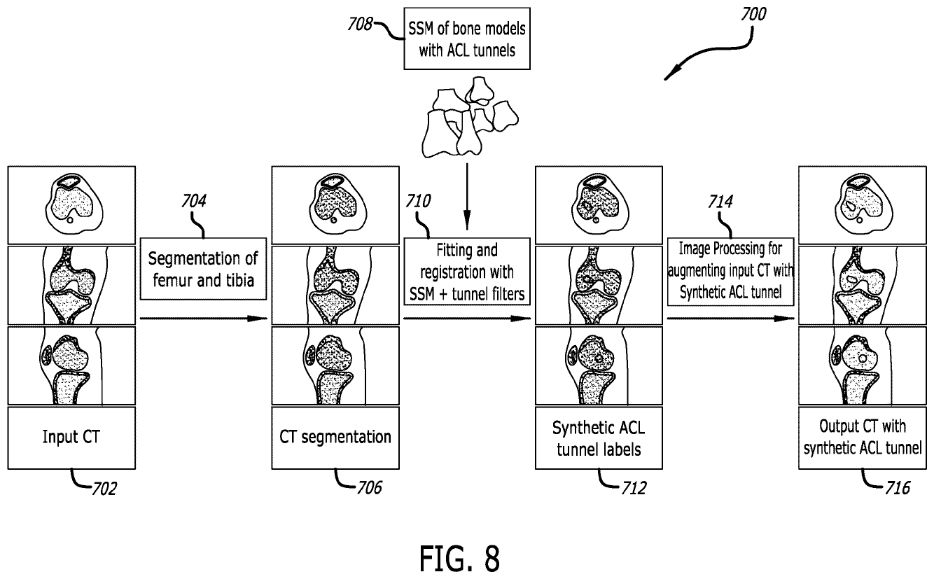

Turning to FIG. 8, an alternative view of Process 700 is depicted. Steps 702-704 are depicted with corresponding imagery to depict how the input CT image (as depicted in relation to Step 702) is segmented to form a CT segmentation (Steps 704-706), fitted with an identified SSM model (Steps 708-712), whereby an augmented CT image is output, which includes synthetic ACL tunnels (Steps 714-716).

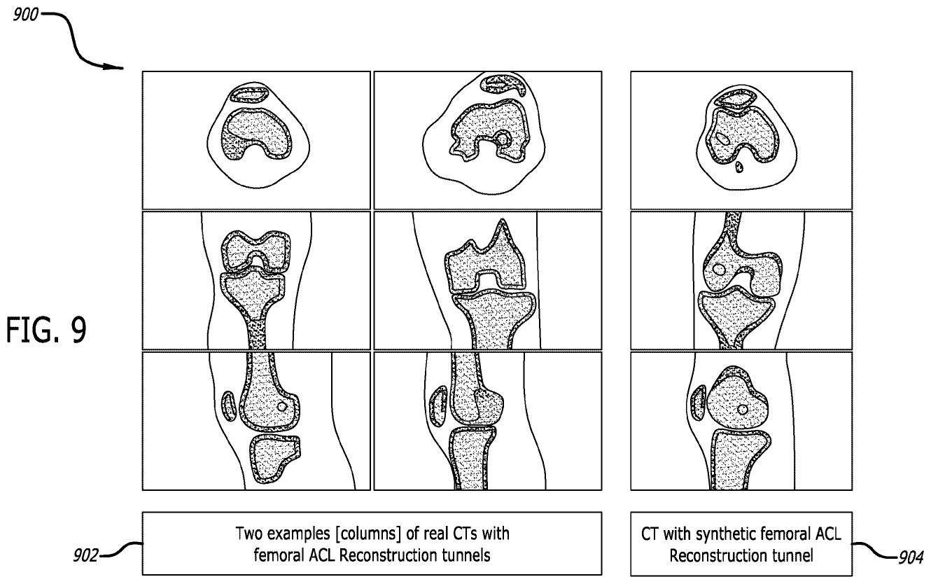

Turning to FIG. 9, depicted is example comparison 900, which showcases the differences between CT images 902, which have ACL reconstruction tunnels, and CT images 904, which have synthetic ACL tunnels.

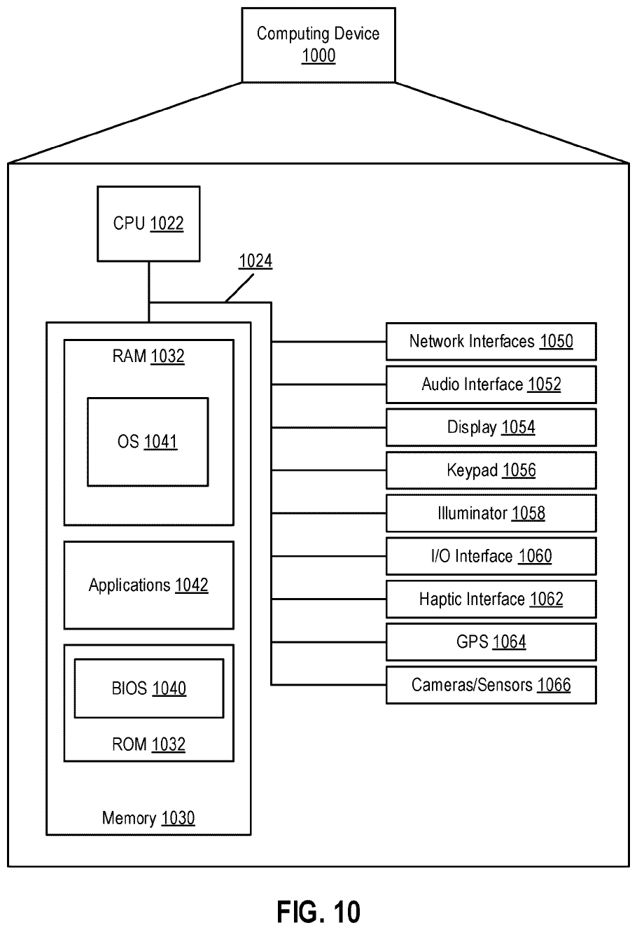

FIG. 10 is a block diagram illustrating a computing device 1000 (e.g., UE 1000, as discussed above) showing an example of a client device or server device used in the various embodiments of the disclosure.

The computing device 1000 may include more or fewer components than those shown in FIG. 10, depending on the deployment or usage of the device 1000. For example, a server computing device, such as a rack-mounted server, may not include audio interfaces 1052, displays 1054, keypads 1056, illuminators 1058, haptic interfaces 1062, GPS receivers 1064, or cameras/sensors 1066. Some devices may include additional components not shown, such as GPU devices, cryptographic co-processors, AI accelerators, or other peripheral devices.

As shown in FIG. 10, the device 1000 includes a central processing unit (CPU) 1022 in communication with a mass memory 1030 via a bus 1024. The computing device 1000 also includes one or more network interfaces 1050, an audio interface 1052, a display 1054, a keypad 1056, an illuminator 1058, an input/output interface 1060, a haptic interface 1062, an optional GPS receiver 1064 (and/or an interchangeable or additional GNSS receiver) and a camera(s) or other optical, thermal, or electromagnetic sensors 1066. Device 1000 can include one camera/sensor 1066 or a plurality of cameras/sensors 1066. The positioning of the camera(s)/sensor(s) 1066 on the device 1000 can change per device 1000 model, per device 1000 capabilities, and the like, or some combination thereof.

In some embodiments, the CPU 1022 may comprise a general-purpose CPU. The CPU 1022 may comprise a single-core or multiple-core CPU. The CPU 1022 may comprise a system-on-a-chip (SoC) or a similar embedded system. In some embodiments, a GPU may be used in place of, or in combination with, a CPU 1022. Mass memory 1030 may comprise a dynamic random-access memory (DRAM) device, a static random-access memory device (SRAM), or a Flash (e.g., NAND Flash) memory device. In some embodiments, mass memory 1030 may comprise a combination of such memory types. In one embodiment, the bus 1024 may comprise a Peripheral Component Interconnect Express (PCIe) bus. In some embodiments, the bus 1024 may comprise multiple busses instead of a single bus.

Mass memory 1030 illustrates another example of computer storage media for the storage of information such as computer-readable instructions, data structures, program modules, or other data. Mass memory 1030 stores a basic input/output system (“BIOS”) 1040 for controlling the low-level operation of the computing device 1000. The mass memory also stores an operating system 1041 for controlling the operation of the computing device 1000.

Applications 1042 may include computer-executable instructions which, when executed by the computing device 1000, perform any of the methods (or portions of the methods) described previously in the description of the preceding Figures. In some embodiments, the software or programs implementing the method embodiments can be read from a hard disk drive (not illustrated) and temporarily stored in RAM 1032 by CPU 1022. CPU 1022 may then read the software or data from RAM 1032, process them, and store them to RAM 1032 again.

The computing device 1000 may optionally communicate with abase station (not shown) or directly with another computing device. Network interface 1050 is sometimes known as a transceiver, transceiving device, or network interface card (NIC).

The audio interface 1052 produces and receives audio signals such as the sound of a human voice. For example, the audio interface 1052 may be coupled to a speaker and microphone (not shown) to enable telecommunication with others or generate an audio acknowledgment for some action. Display 1054 may be a liquid crystal display (LCD), gas plasma, light-emitting diode (LED), or any other type of display used with a computing device. Display 1054 may also include a touch-sensitive screen arranged to receive input from an object such as a stylus or a digit from a human hand.

Keypad 1056 may comprise any input device arranged to receive input from a user. Illuminator 1058 may provide a status indication or provide light.

The computing device 1000 also comprises an input/output interface 1060 for communicating with external devices, using communication technologies, such as USB, infrared, Bluetooth™, or the like. The haptic interface 1062 provides tactile feedback to a user of the client device.

The optional GPS transceiver 1064 can determine the physical coordinates of the computing device 1000 on the surface of the Earth, which typically outputs a location as latitude and longitude values. GPS transceiver 1064 can also employ other geo-positioning mechanisms, including, but not limited to, triangulation, assisted GPS (AGPS), E-OTD, CI, SAI, ETA, BSS, or the like, to further determine the physical location of the computing device 1000 on the surface of the Earth. In one embodiment, however, the computing device 1000 may communicate through other components, provide other information that may be employed to determine a physical location of the device, including, for example, a MAC address, IP address, or the like.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

1. A method comprising:
identifying, by a device, an image associated with a knee, the image depicting at least a portion of a femur and tibia after an initial anterior cruciate ligament (ACL) reconstruction procedure;
analyzing, by the device, the image, and performing a first segmentation of the image, the first segmentation comprising information related to the femur and tibia, the first segmentation further comprising information related to tunnels associated with the initial ACL reconstruction procedure;
further analyzing, by the device, the image, and performing a second segmentation of the image, the second segmentation comprising information related to hardware associated with the initial ACL reconstruction procedure; and
generating, by the device, a three-dimensional (3D) model of the knee based on the first segmentation and the second segmentation.

2. The method of claim 1, further comprising:
performing, by the device, the second segmentation according to a predetermined range of Hounsfield Units (HU), the predetermined range corresponding to and enabling identification of a presence of a particular type of material from the image.

3. The method of claim 1, wherein the further analysis related to the second segmentation further comprises a thresholding operation.

4. The method of claim 1, further comprising:
providing, by the device, the first segmentation and the second segmentation as input into a medical imaging interaction toolkit (MITK) software application; and
executing, by the device, the MITK software application, wherein the generation of the 3D model is based on the execution of the MITK software.

5. The method of claim 1, further comprising:
generating, by the device, an operative plan for an ACL revision procedure based on the generated 3D model.

6. The method of claim 1, wherein the hardware corresponds to a set of screws used as part of the initial ACL reconstruction procedure.

7. The method of claim 1, wherein the image is at least one selected from a group comprising: a computed tomography (CT) image; and a magnetic resonance imaging (MRI) image.

8. (canceled)

9. A non-transitory computer-readable storage medium tangibly encoded with computer-executable instructions, that when executed by a device, perform a method comprising:
identifying, by the device, an image associated with a knee, the image depicting at least a portion of a femur and tibia after an initial anterior cruciate ligament (ACL) reconstruction procedure;
analyzing, by the device, the image, and performing a first segmentation of the image, the first segmentation comprising information related to the femur and tibia, the first segmentation further comprising information related to tunnels associated with the initial ACL reconstruction procedure;
further analyzing, by the device, the image, and performing a second segmentation of the image, the second segmentation comprising information related to hardware associated with the initial ACL reconstruction procedure; and
generating, by the device, a three-dimensional (3D) model of the knee based on the first segmentation and the second segmentation.

10. The non-transitory computer-readable storage medium of claim 9, further comprising:
performing, by the device, the second segmentation according to a predetermined range of Hounsfield Units (HU), the predetermined range corresponding to and enabling identification of a presence of a particular type of material from the image.

11. The non-transitory computer-readable storage medium of claim 9, wherein the further analysis related to the second segmentation further comprises a thresholding operation.

12. The non-transitory computer-readable storage medium of claim 9, further comprising:
providing, by the device, the first segmentation and the second segmentation as input into a medical imaging interaction toolkit (MITK) software application; and
executing, by the device, the MITK software application, wherein the generation of the 3D model is based on the execution of the MITK software.

13. The non-transitory computer-readable storage medium of claim 9, further comprising:
generating, by the device, an operative plan for an ACL revision procedure based on the generated 3D model.

14. The non-transitory computer-readable storage medium of claim 9, wherein the hardware corresponds to a set of screws used as part of the initial ACL reconstruction procedure.

15. The non-transitory computer-readable storage medium of claim 9, wherein the image is a computed tomography (CT) image.

16. The non-transitory computer-readable storage medium of claim 9, wherein the image is a magnetic resonance imaging (MRI) image.

17. A device comprising:
a processor configured to:
identify an image associated with a knee, the image depicting at least a portion of a femur and tibia after an initial anterior cruciate ligament (ACL) reconstruction procedure;
analyze the image, and perform a first segmentation of the image, the first segmentation comprising information related to the femur and tibia, the first segmentation further comprising information related to tunnels associated with the initial ACL reconstruction procedure;
further analyze the image, and perform a second segmentation of the image, the second segmentation comprising information related to a location of hardware associated with the initial ACL reconstruction procedure; and
generate a three-dimensional (3D) model of the knee based on the first segmentation and the second segmentation.

18. The device of claim 17, wherein the processor is further configured to:
perform the second segmentation according to a predetermined range of Hounsfield Units (HU), the predetermined range corresponding to and enabling identification of a presence of a particular type of material from the image.

19. The device of claim 17, wherein the processor is further configured to:
provide the first segmentation and the second segmentation as input into a medical imaging interaction toolkit (MITK) software application; and
execute the MITK software application, wherein the generation of the 3D model is based on the execution of the MITK software.

20. The device of claim 17, wherein the processor is further configured to:
generate an operative plan for an ACL revision procedure based on the generated 3D model.

21. A method comprising:
identifying, by a device, an image associated with a knee, the image depicting at least a portion of a femur and tibia;
analyzing, by the device, the image, and identifying information related to the femur and tibia;
generating, by the device, based on the analysis, a segmentation image, the segmentation image comprising a depiction of the femur and tibia;
identifying, by the device, a bone model for another knee, the bone model comprising information related to a femur, tibia and tunnels corresponding to an initial anterior cruciate ligament (ACL) reconstruction procedure;
performing fitting, by the device, of the bone model to the segmentation image, the fitting comprising identification of the tunnels in relation to the femur and tibia of the segmentation image; and
generating, by the device, a synthetic image based on the fitting of the bone model.

22.-40. (canceled)
